(19) United States
(12) Patent Application Publication
(10) Pub. No.: US 2026/0248729 A1
(43) Pub. Date: Aug. 27, 2026

(54) NOVEL ORAL DOSAGE FORM

(71) Applicant: Neon Healthcare Ltd, Hertford (GB)

(72) Inventors: Stephen Knightley, Hertford (GB); Dharmik Patel, Hertford (GB); Bikrum Prem, Hertford (GB)

(21) Appl. No.: 18/875,924

(22) PCT Filed: Jun. 20, 2023
(86) PCT No.: PCT/GB2023/051612

§ 371 Date: Dec. 17, 2024

(30) Foreign Application Priority Data
Jun. 20, 2022 (GB) 2209047.6

(51) Int. Cl. A61K 9/28; A61K 9/00; A61K 9/20; A61K 9/48; A61K 31/15

(52) U.S. Cl. CPC A61K 9/284; A61K 9/0053; A61K 9/2018; A61K 9/2027; A61K 9/2059; A61K 9/2813; A61K 9/282; A61K 9/4858; A61K 9/4866; A61K 9/4891; A61K 31/15

(57) ABSTRACT

The invention relates to an oral dosage form comprising phenelzine and a film coating, characterised in that said film coating is an aqueous based film coating. The invention also relates to said oral dosage form for use in the treatment of depression (such as major depressive disorder (MDD)), anxiety, or a disorder related to depression or anxiety.

### FIELD OF THE INVENTION

The invention relates to an oral dosage form comprising phenelzine and a film coating, characterised in that said film coating is an aqueous based film coating. The invention also relates to said oral dosage form for use in the treatment of depression (such as major depressive disorder (MDD)), anxiety, or a disorder related to depression or anxiety.

### BACKGROUND OF THE INVENTION

Phenelzine has the chemical formula:

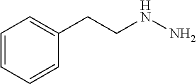

and its synthesis was first described in 1932 by Emil Votoček and Otakar Leminger. Phenelzine (also known as 2-phenylethylhydrazine) can be synthesized by reacting 2-phenylethylbromide with hydrazine. An example of a large scale synthesis of phenelzine sulfate is possible from phenethylamine and 3,3-pentamethylene oxaziridine in the presence of an acid catalyst. Phenelzine is known to be a non-selective and irreversible monoamine oxidase inhibitor (MAOI) of the hydrazine class. Phenelzine sulfate has known properties as an anti-depressant and anxiolytic.

Phenelzine is administered orally in the form of phenelzine sulfate and is rapidly absorbed from the gastrointestinal tract. Time to peak plasma concentration is 43 minutes and half-life is 11.6 hours. Unlike most other drugs, phenelzine irreversibly disables MAO, and as a result, it does not necessarily need to be present in the blood at all times for its effects to be sustained. Because of this, upon phenelzine treatment being ceased, its effects typically do not actually wear off until the body replenishes its enzyme stores, a process which can take as long as 2-3 weeks.

Phenelzine is metabolized primarily in the liver and its metabolites are excreted in the urine. Oxidation is the primary routine of metabolism, and the major metabolites are phenylacetic acid and parahydroxyphenylacetic acid, recovered as about 73% of the excreted dose of phenelzine in the urine over the course of 96 hours after single doses. Acetylation to N2-acetylphenelzine is a minor pathway. Phenelzine may also interact with cytochrome P450 enzymes, inactivating these enzymes through formation of a heme adduct. Two other minor metabolites of phenelzine, include phenylethylidenehydrazine and phenethylamine (PEA).

PEA does not have any obvious, easily discernible, reliably induced effects when administered to humans. Phenelzine's enhancement of PEA levels may contribute further to its overall antidepressant effects to some degree. In addition, phenethylamine is a substrate for MAO-B, and treatment with MAOIs that inhibit MAO-B such as phenelzine have been shown to consistently and significantly elevate its concentrations.

Like many other antidepressants, phenelzine usually requires several weeks of treatment to achieve full therapeutic effects. The reason for this delay is not fully understood, but it is believed to be due to many factors, including achieving steady-state levels of MAO inhibition and the resulting adaptations in mean neurotransmitter levels, the possibility of necessary desensitization of autoreceptors which normally inhibit the release of neurotransmitters like serotonin and dopamine, and also the upregulation of enzymes such as serotonin N-acetyltransferase. Typically, a therapeutic response to MAOls is associated with an inhibition of at least 80-85% of monoamine oxidase activity.

In 2019, unspecified manufacturing issues resulted in a significant shortage of availability of Phenelzine which impacted a large number of patients who relied upon this drug for managing depression. It was even speculated that the worldwide shortage of this drug product would “kill people” (https://medium.com/@peterjeffers36/a-shortage-of-this-antidepressant-will-kill-people-818c52732010).

Thus, there is a great need for an alternative dosage form of phenelzine which can be manufactured in a safe, controlled manner and which results in a stable product.

### SUMMARY OF THE INVENTION

According to a first aspect of the invention, there is provided an oral dosage form comprising:
(a) a pharmaceutical composition comprising phenelzine or a pharmaceutically acceptable salt thereof, in combination with one or more pharmaceutically acceptable excipients; and
(b) a film coating surrounding said pharmaceutical composition;
   characterised in that said film coating is an aqueous based film coating.

According to a second aspect of the invention, there is provided the oral dosage form of the invention for use in the treatment of depression, anxiety, or a disorder related to depression or anxiety.

### BRIEF DESCRIPTION OF THE FIGURES

FIG. 1: Flow Diagram of Manufacturing Process.

### DETAILED DESCRIPTION OF THE INVENTION

According to a first aspect of the invention, there is provided an oral dosage form comprising:
(a) a pharmaceutical composition comprising phenelzine or a pharmaceutically acceptable salt thereof, in combination with one or more pharmaceutically acceptable excipients; and
(b) a film coating surrounding said pharmaceutical composition;
   characterised in that said film coating is an aqueous based film coating.

The finished product was historically manufactured using a solvent based coating. By contrast, the present invention requires the coating to be an aqueous one. To date, an aqueous based moisture barrier film coating on a phenelzine tablet has not been developed/invented. The resultant composition is one which alleviates health and safety concerns during the manufacturing process. In addition, an aqueous based, high performance moisture barrier film coating helps to protect the dosage form against moisture and improve the overall stability of the dosage form. It ensures the phenelzine dose is administered correctly within the human. Furthermore, the combination of the coating ingredients with a phenelzine dosage form ensures manufacture of a stable product. The film coating protects the tablet from moisture, improving the overall stability and shelf-life of the finished drug product. For example, data is presented herein which confirms that the oral dosage form of the invention is stable in refrigerated conditions and additionally has an in-use shelf life of up to 200 days which supports the use throughout a patients treatment (see Example 2). Furthermore, data is also presented herein which confirms that the aqueous coating of the oral dosage form is compatible with the phenelzine active and allows the phenelzine tablet to be stable for up to 24 months (see Example 1). The solvent coating presents risks to overall product quality, potentially patient safety, the environment and employee health and safety (at the manufacturing site). The aqueous coating eliminates these risk factors, whilst maintaining the quality of the dosage form. The dosage form of the invention also ensures the key active ingredient is delivered to the patient effectively as there is minimal impact to performance with respect to dissolution or disintegration time, data supports that the invention has a better dissolution profile.

### Oral Dosage Forms

It will be appreciated that the oral dosage form of the invention can be any suitable dosage form which is capable of administration via the oral route. In one embodiment, the oral dosage form is a solid, oral dosage form. Examples of sold oral dosage forms include tablets and capsules. In one particular embodiment of the invention, the oral dosage form is a tablet.

### Pharmaceutically Acceptable Salts

In one embodiment, the phenelzine exists as a pharmaceutically acceptable salt. In a further embodiment, the phenelzine exists as a pharmaceutically acceptable acid addition salt.

Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of acid addition salts consists of salts formed from acetic, hydrochloric, hydriodic, phosphoric, nitric, sulfuric, citric, lactic, succinic, maleic, malic, isethionic, fumaric, benzenesulfonic, toluenesulfonic, methanesulfonic (mesylate), ethanesulfonic, naphthalenesulfonic, valeric, acetic, propanoic, butanoic, malonic, glucuronic and lactobionic acids.

In one particular embodiment of the invention, the phenelzine exists as the sulfate salt.

The preparation of phenelzine sulfate will be well known to the skilled person and could typically involve mixing phenelzine in a suitable solvent (such as isopropyl alcohol) with stirring at a suitable temperature (such as 0-5° C.) followed by addition of sulfuric acid in a suitable solvent (such as isopropyl alcohol) followed by stirring and addition of a further suitable solvent (such as n-heptane) followed by suitable extraction of the resultant salted product (such as filtration and washing in a suitable solvent, such as isopropyl alcohol).

The phenelzine sulfate is typically present within the oral dosage form in a range of between 10 mg and 90 mg, e.g. between 20 mg and 30 mg, such as between 22 mg and 28 mg, in particular between 24.93 mg and 27.12 mg, more particularly about 26 mg, especially 25.83 mg per oral dosage form.

### Pharmaceutically Acceptable Excipients

The pharmaceutically acceptable excipient(s) can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions. Examples of excipients for various types of pharmaceutical compositions are set out in more detail below.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Pharmaceutical compositions containing phenelzine are formulated based on market regulatory guidance and in accordance with current Good Manufacturing Practice.

In one embodiment, the pharmaceutical composition comprises a diluent. Diluents act as fillers in pharmaceutical tablets to increase weight and improve content uniformity. Natural diluents include starches, hydrolyzed starches, and partially pregelatinized starches. Diluents provide better tablet properties such as improved cohesion or to promote flow. Diluents must be non-toxic, commercially available in acceptable grade, physiologically inert, and physically and chemically stable by themselves as well as in combination with active pharmaceutical ingredients (APIs). Examples of suitable diluents include: anhydrous lactose, lactose monohydrate, and sugar alcohols such as sorbitol, xylitol and mannitol. In one particular embodiment, the diluent is mannitol. Mannitol provides the advantage of having a desirable sensation when it is used in chewable tablets. Mannitol is typically present within the oral dosage form in a range of between 150 mg and 250 mg, e.g. between 175 mg and 200 mg, such as between 179.45 mg and 195.26 mg, more particularly approximately 186 mg, most particularly 185.96 mg per oral dosage form.

In one embodiment, the pharmaceutical composition comprises a disintegrant. A disintegrant is an agent, used in the preparation of tablets, which causes them to disintegrate and release their medicinal substances on contact with moisture. In general, disintegrants help a tablet to break up after oral administration. Examples of suitable disintegrants include: traditional disintegrants, such as starch, microcrystalline cellulose, and sodium alginate etc; and super disintegrants, such as crospovidone (cross-linked povidone), croscarmellose sodium (cross-linked cellulose) and sodium starch glycolate (cross-linked starch) etc. In one particular embodiment, the disintegrant is povidone (polyvinylpyrrolidone, PVP). The presence of a povidone as a disintegrant provides the advantage of being easily dissolved in solvent. Povidone is typically present within the oral dosage form in a range of between 1 mg and 10 mg, such as between 5 mg and 8 mg, more particularly approximately 7 mg, most particularly 6.83 mg per oral dosage form.

In one embodiment, the pharmaceutical composition comprises a lubricant. Lubricants prevent adherence of granule/powder to punch die/faces and promote smooth ejection from the die after compaction. Lubricants can also be used when compression isn't involved such as in powder blends for filling into capsules to prevent adherence of granule/powder to equipment surfaces and dosator mechanisms and coating the surface of multi-particulate dosage forms to inhibit agglomeration of individual particles. The most widely used lubricants in use today are hydrophobic lubricants. These are usually effective at relatively low concentrations and many also have both anti-adherent and glidant properties. Examples of suitable lubricants include: minerals such as talc or silica, and fats, e.g. vegetable stearin, magnesium stearate or stearic acid. In one particular embodiment, the lubricant is magnesium stearate. Magnesium stearate provides the advantage of being capable of forming films on other tablet excipients during prolonged mixing, leading to a prolonged drug liberation time, a decrease in hardness, and an increase in disintegration time. Magnesium stearate is typically present within the oral dosage form in a range of between 1 mg and 10 mg, e.g. between 5 mg and 10 mg, such as between 3 mg and 8 mg, more particularly between 6.57 mg and 7.1 mg, most particularly approximately 7 mg, especially 6.81 mg per oral dosage form.

In one embodiment, the pharmaceutical composition comprises a binder. Binder excipients are formulated to act as an adhesive to literally “bind together” powders, granules and other dry ingredients to impart to the product the necessary mechanical strength. They can also give volume to low active dose tablets. Commonly used in wet granulation, binders are added to create a more effective and predictable granule formation. Binders are classified according to their application. Examples of suitable binders include: gelatin, cellulose, cellulose derivatives, polyvinylpyrrolidone, starch (such as maize starch), sucrose and polyethylene glycol. In one particular embodiment, the binder is starch, such as maize starch. Maize starch is typically present within the oral dosage form in a range of between 1 mg and 10 mg, such as between 2 mg and 8 mg, more particularly approximately 5 mg, most particularly 4.57 mg per oral dosage form.

It will be appreciated that the pharmaceutical composition is typically formulated in the presence of a suitable solvent. In one particular embodiment, the solvent is isopropyl alcohol.

### Film Coating

It will be appreciated that the film coating represents the key inventive feature of the oral dosage form based on the requirement for the film coating to be an aqueous based film coating. It will be apparent to the skilled person that the aqueous film coating may be prepared by combining the coating ingredients described herein with water as the solvent rather than requiring the use of an alcohol based solvent system. Due to the difference of solvent used, the composition of the coating will also differ to have components which are soluble in the respective solvent.

In one embodiment, the film coating comprises the following components: polyvinyl alcohol, talc, glyceryl dicaprylocaprate, glyceryl monocaprylocaprate, sodium lauryl sulfate, and titanium dioxide.

Typically, the polyvinyl alcohol is present within the film coating in an amount of between 1 mg and 10 mg, such as between 4 mg and 6 mg, in particular approximately 5 mg, most particularly 5.45 mg per oral dosage form.

Typically, the talc is present within the film coating in an amount of between 1 mg and 10 mg, such as between 5 mg and 7 mg, in particular approximately 5 mg, most particularly 5.24 mg per oral dosage form.

Typically, the combination of glyceryl dicaprylocaprate and glyceryl monocaprylocaprate is present within the film coating in an amount of between 0.5 mg and 5 mg, such as between 0.5 mg and 1 mg, in particular approximately 0.5 mg, most particularly 0.62 mg per oral dosage form.

Typically, the sodium lauryl sulfate is present within the film coating in an amount of between 0.01 mg and 2 mg, e.g. between 0.1 mg and 1 mg, such as between 0.2 mg and 0.6 mg, in particular approximately 0.4 mg, most particularly 0.41 mg per oral dosage form.

Typically, the titanium dioxide is present within the film coating in an amount of between 0.1 mg and 1 mg, such as between 0.2 mg and 0.6 mg, in particular approximately 0.4 mg, most particularly 0.41 mg per oral dosage form.

In one embodiment, the film coating is also required to be a coloured coating. In a further embodiment, the coating is a yellow to red (i.e. orange) colour. In a yet further embodiment, the coating is an orange colour. This orange coloured embodiment has the advantage of providing a drug product which allows continuity of supply to patients, in particular for the UK and global developed markets. As this coating is similar in colour to the previously registered solvent based coating, it will be familiar to the end user patient population. This is an important factor given the intended target patient population based on the therapeutic indication of the product. Historically the product has been orange in colour and due to the nature of the indication, this is important to the patient population as it provides familiarity and reassurance to patients regarding the medication they are taking. To achieve the correct orange colouring of the film coating, the coating additionally comprises a combination of dyes, such as sunset yellow (i.e. sunset yellow FCF aluminium lake, E110) and carmine (E120). To achieve the correct orange colour, the sunset yellow is typically present within the film coating in an amount of between 1 mg and 5 mg, such as between 0.5 mg and 3 mg, in particular approximately 2 mg, more particularly 1.52 mg per oral dosage form and the carmine is typically present within the film coating in an amount of between 0.01 mg and 2 mg, such as between 0.1 mg and 0.3 mg, in particular approximately 0.1 mg, more particularly 0.14 mg per oral dosage form.

The resultant film coating is provided within the oral dosage form in an amount of between 5 mg and 25 mg, e.g. between 10 mg and 20 mg, such as between 12 mg and 18 mg, in particular between 13.8 mg and 16.56 mg, more particularly approximately 14 mg, especially 13.80 mg per oral dosage form.

### Therapeutic Uses

As phenelzine has been readily prescribed for the treatment of depression and anxiety since approval in 1961 it will be appreciated that the oral dosage form of the invention finds utility in the treatment of such disorders. Thus, according to a second aspect of the invention, there is provided the oral dosage form of the invention for use in the treatment of depression, anxiety, or a disorder related to depression or anxiety.

In one particular embodiment, the depression is major depressive disorder (MDD).

Examples of disorders related to depression or anxiety include: dysthymia, bipolar depression (BD), panic disorder (PD), social anxiety disorder, bulimia, post-traumatic stress disorder (PTSD), and obsessive-compulsive disorder (OCD).

The oral dosage form of the invention is generally administered to a subject in need of such administration, for example a human or animal patient, particularly a human.

In one particular embodiment, the dosage form is administered to a subject who has been clinically characterised with atypical, non-endogenous or neurotic depression or where treatment with other antidepressants has failed. Such patients often have mixed anxiety and depression and phobic or hypochondriacal features.

According to a further aspect of the invention is provided the use of a dosage form of the invention in the treatment of depression, anxiety, or a disorder related to depression or anxiety.

According to a further aspect of the invention is provided the use of a dosage form of the invention in the manufacture of a medicament for the treatment of depression, anxiety, or a disorder related to depression or anxiety.

According to a further aspect of the invention is provided a method of treating depression, anxiety, or a disorder related to depression or anxiety, wherein said method comprises administering a subject in need thereof the dosage form of the invention.

As phenelzine has been readily prescribed for the treatment of depression and anxiety since approval in 1961, it will be appreciated that a suitable dosage regime for the claimed disorders is well known. For example, such a dosage regime for a starting dose would typically be one 15 mg tablet three times per day. Dosage should then be increased to at least 60 mg per day (i.e. up to 90 mg per day) for early phase treatment. Once the clinical benefit of phenelzine has been achieved, dosage should be reduced slowly over several weeks with a maintenance dose which would typically be as low as one 15 mg tablet once per day or every other day, continued as long as required.

### EXAMPLES

One specific example of the oral dosage form of the invention is shown in Tables 1 and 2 below which constitutes a specifically preferred embodiment of the invention.

TABLE 1
Composition of Drug Product

| | | | |
|---|---|---|---|
| | Quantity | | |
| Ingredient | (mg/tablet) | Function | Standard |

Active Substance

| | | | |
|---|---|---|---|
| Phenelzine Sulfate | 25.83 | Active | BP |
| (Equivalent to | | | |
| Phenelzine base | | | |
| 15 mg) | | | |

Excipients

| | | | |
|---|---|---|---|
| Mannitol | 185.96 | Diluent | Ph. Eur. |
| Povidone | 6.83 | Disintegrant | Ph. Eur. |
| Magnesium Stearate | 6.81 | Lubricant | Ph. Eur. |
| Maize Starch | 4.57 | Binder | Ph. Eur. |
| Isopropyl alcohol | ND | Solvent | Ph. Eur. |

Film Coating

| | | | |
|---|---|---|---|
| Opadry AMB II Orange | 13.80 | Coating | See Table |
| | | | 2 |
| Water Purified | ND | Solvent | Ph. Eur. |

TABLE 2
Composition of Opadry AMB II Orange

| | Quantity | % | | Reference |
|---|---|---|---|---|
| Ingredient | (mg/tablet) | w/w | Function | Standard |
| Polyvinyl Alcohol | 5.45 | 39.5 | Coating | USP, FCC, Ph. Eur, |
| | | | | JPE, ChP, GB |
| Talc | 5.24 | 38.0 | Coating | USP, FCC, Ph. |
| | | | | Eur, JP, JECFA |
| Sunset Yellow FCF | 1.52 | 11.0 | Coating | JECFA, JSFA, |
| Aluminum Lake | | | | JP MO, GB |
| Glyceryl | 0.62 | 4.5 | Coating | NF, Glycerol of |
| Dicaprylocaprate | | | | FA JSFA |
| Glyceryl | | | | Type 1 Ph. |
| Mono- | | | | Eur, Glycerol |
| caprylocaprate | | | | of FA JSFA |
| Sodium Lauryl | 0.41 | 3.0 | Coating | NF, Ph. Eur, |
| Sulfate | | | | JP, ChP 2015 |
| Titanium Dioxide | 0.41 | 3.0 | Coating | USP, FCC, Ph. |
| | | | | Eur, JP, |
| | | | | JSFA, ChP, GB |
| Carmine | 0.14 | 1.0 | Coating | FCC, JPE, JECFA |

Phenelzine tablets are smooth, orange, uniformly coated, biconvex tablets with rounded edges, and having not more than a slight odour. The product is contained in a white plastic bottle within a cardboard carton.

### Description of Manufacturing Process

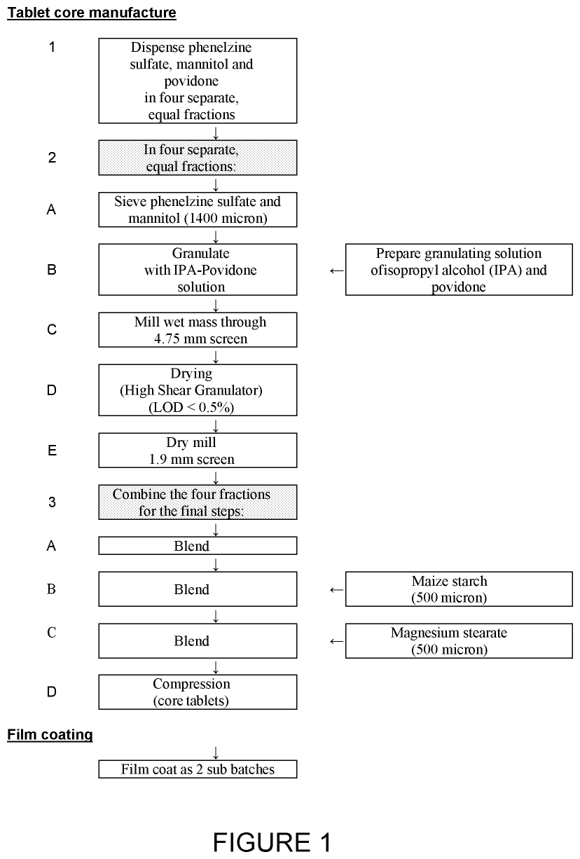

An overview of the manufacturing process is provided in FIG. 1 and further specific details are provided below.

### Tablet Core Manufacture

1. Dispense phenelzine sulfate, mannitol and povidone into four separate, equal fractions.
2. Complete the following steps in four separate equal fractions:
   A. Sieve the phenelzine sulfate and mannitol through a 1400 micron sieve.
   B. Dissolve the povidone in isopropyl alcohol. Add to the powder from Step 2A. Mix for approximately 11 minutes at 200 rpm until the required granulation is obtained. Add extra isopropyl alcohol and mix again if necessary.
   C. Mill the wet mass through a 4.75 mm screen.
   D. Dry the granulate in a high shear granulator at 55° C. (50 rpm) until the Loss on Drying (LOD) limit of less than 0.5% is achieved.
   E. Mill the dried granulate through a 1.9 mm screen.
3. Combine the four fractions for the final steps below:
   A. Blend the four fractions of dried granule for approximately 5 minutes at 15 rpm.
   B. Sieve the maize starch through a 500 micron sieve and blend with the dried granules for approximately 15 minutes at 15 rpm.
   C. Sieve the magnesium stearate through a 500 micron sieve and blend with the material from Step 3B for approximately 3 minutes at 15 rpm.
   D. Compress tablets.

### Film Coating

1. Film coat the tablet cores with a previously dispersed suspension of Opadry AMB II Orange and 9539 g of purified water. The coating process is performed in two equal fractions to ensure even coating.

### Packaging

The approved product is packaged using the specified packaging material.

According to a further aspect of the invention there is provided a process for preparing the oral dosage form of the invention which comprises the steps described hereinbefore and/or in FIG. 1.

### Data

A range of stability analyses were conducted on the oral dosage form of the invention which are detailed herein:

### Example 1: Drug Product Stability Analysis

### 1 Analytical Procedures

### 1.1 Description

Examine the tablets critically under normal laboratory light. Inspect the tablets to assure that all characteristics indicated in the specifications conform to the criteria.

### 1.2 Colour

Examine the tablets critically under normal laboratory light. Inspect for colour (uniformity and presence/absence of discolouration).

### 1.3 Coating

Examine the tablets critically under normal laboratory light. Inspect for presence/absence of defects.

### 1.4 Disintegration

The test to be performed as per Ph. Eur. 2.9.1. Disintegration to be performed on 6 tablets in purified water at 37±2° C. without plastic discs.

### 1.5 Loss on Drying

The test to be performed as per Ph. Eur. 2.2.32. Loss on drying to be performed on 1 g sample (from 6 ground tablets) at 105° C.

### 1.6 Assay—Phenelzine

### Reagents

Purified water
Methanol
Sodium 1-octanesulfonate
Monobasic potassium phosphate
Orthophosphoric acid 85%

Ion-pair solution: Dissolve 6.8 g of monobasic potassium phosphate and 2.16 g of sodium 1-octanesulfonate in 1000 ml of water. Adjust to pH 3.0±0.1 with orthophosphoric acid.

### HPLC Conditions

| Column: | Symmetry C18 5 | |
|---|---|---|
| Guard Column: | Security Guard C18/, | |
| Mobile phase: | Ion-pair solution: | |
| Flow rate: | 1.0 | ml/minute |
| Column temperature: | 30° | C. |
| Detection: | UV at 210 nm | |
| Injection: | 20 | μl |
| Run time: | 20 | minutes |

### Standard Solution (Prepare in Duplicate)

Accurately weigh approximately 25.80 mg of phenelzine sulfate reference standard into a 100 ml volumetric flask. Dissolve in approximately 80 ml of mobile phase. Equilibrate to room temperature, dilute to volume with mobile phase and mix.

### Sample Preparation (Prepare in Duplicate)

Transfer 20 tablets to a 500 ml volumetric flask, add approximately 300 ml of mobile phase. Sonicate and shake mechanically until fully disintegrated. Equilibrate to room temperature, dilute with mobile phase to volume and mix. Centrifuge the solution or alternatively filter through a 0.45 μm RC filter. Transfer 5 ml of the solution to a 20 ml volumetric flask, dilute with mobile phase to volume and mix.

### Procedure

Inject samples and standard into an HPLC using the conditions described above.

### System Suitability Criteria

| Parameter | Specification |
|---|---|
| Tailing factor (Calculate for first reference standard injection) | ≤2.0 |
| Standard Verification | 98.0%-102.0% |
| % RSD of peak area responses for the 5 reference standard A | ≤2.0% |
| injections | |
| % RSD for peak area responses for bracketing standard | ≤2.0% |
| injections throughoutrun | |
| % RSD for retention times for the 5 reference standard A | ≤1.0% |
| injections | |

### Calculation

$$mg per tablet = \frac{PA ( spl )}{PA ( std )} \times \frac{Std Wgt ( mg )}{100 ( mL )} \times \frac{500 ( mL )}{Spl Wgt} \times \frac{20 ( mL )}{5 ( mL )} \times \frac{136.19}{234.28} \times \frac{P}{100}$$

where:

| | |
|---|---|
| PA(spl): | Peak area of Phenelzine in the Sample Chromatogram. |
| PA(std): | Mean Peak area of Phenelzine in the Bracketing |
| | Standard Chromatograms. |
| Std Wgt | Standard weight of Phenelzine Sulphate Reference |
| | Standard (mg). |
| Spl Wgt: | Sample weight (mg). |
| ACW: | Average Capsule Weight |
| P: | Potency of Phenelzine Reference Standard |

### 1.7 Identification

### 1.7.1 HPLC

Using the method described in Section 1.6, identify the phenelzine peak in the respective Sample preparation by comparison with the retention time of the principal peak in the Standard preparation chromatogram. The retention time Ratio should be within 1.00±0.05.

The identification is considered as positive if the retention time of the major peak (phenelzine) in the chromatogram of the sample preparation corresponds to that of the standard preparation.

### 1.7.2 Identification Test A (BP)

The test to be performed as per the British Pharmacopeia.

### 1.8 Uniformity of Dosage Units

The analytical procedure is identical to that described for assay above, except for below:

### Sample Preparation

Perform the following on 10 individual coated tablets:

Transfer one pre-weighed tablet (weight recorded for information only) into a 100 ml volumetric flask.

Add approximately 60 ml of Mobile Phase. Sonicate and shake mechanically until the tablets have fully disintegrated. Allow to equilibrate to room temperature. Dilute to volume with Mobile Phase and mix well. Centrifuge the solution or alternatively filter through a 0.45 μm RC filter.

### System Suitability Criteria

| Parameter | Specification |
|---|---|
| Tailing factor (Calculate for first reference standard injection) | ≤2.0 |
| Standard Verification | 98.0%-102.0% |
| % RSD of peak area responses for the 5 reference standard A | ≤2.0% |
| injections | |
| % RSD for peak area responses for bracketing standard | ≤2.0% |
| injections throughoutrun | |
| % RSD for retention times for the 5 reference standard A | ≤1.0% |
| injections | |

### Calculation

$$\% Assay = \frac{PA ( spl )}{PA ( std )} \times \frac{Std Wgt ( mg )}{100 ( mL )} \times \frac{100 ( mL )}{L C} \times \frac{136.19}{234.28} \times \frac{P}{100} \times 100$$

where:

| | |
|---|---|
| PA(spl): | Peak area of Phenelzine in the Sample Chromatogram. |
| PA(std): | Mean Peak area of Phenelzine in the Bracketing |
| | Standard Chromatograms. |
| Std Wgt | Standard weight of Phenelzine Sulphate Reference |
| | Standard (mg). |
| Spl Wgt: | Sample weight (mg). |
| LC: | Label Claim i.e. 15 mg. |
| P: | Potency of Phenelzine Reference Standard |

### 1.9 Related Substances

### HPLC Conditions

| Column | Phenomenex Luna C18 (2), 5 μm | |
|---|---|---|
| | (15 cm × 4.6 mm ID), or equivalent | |
| Guard Column | Security Guard C18, 3 4 mm | |
| | (Phenomenex or equivalent) | |
| Mobile phase | Ion-pair solution: Methanol (60:40 V/V) | |
| Flow rate | 1.4 | ml/minute |
| Column temperature | 20° | C. |
| Detection | UV at 210 nm | |
| Injection | 20 | μl |
| Run time | 25 | minutes |

### Reagents

Purified water
Methanol
Sodium 1-octanesulfonate
Monobasic potassium phosphate
Orthophosphoric acid 85%

Ion-pair solution: Dissolve 6.8 g of monobasic potassium phosphate and 2.16 g of sodium 1-octanesulfonate in 1000 ml of water. Adjust to pH 3.0±0.1 with orthophosphoric acid.

### Standard Solution (Prepare in Duplicate)

Accurately weigh approximately 25.80 mg of phenelzine sulfate reference standard into a 100 ml volumetric flask. Dissolve in approximately 80 ml of mobile phase. Equilibrate to room temperature, dilute to volume with mobile phase and mix. Transfer 2 ml of the standard solution into a 200 ml volumetric flask, dilute to volume with mobile phase and mix well.

### LOQ Preparation

Transfer 10 ml of the Standard Solution into a 100 ml volumetric flask, dilute to volume with mobile phase and mix well.

### Placebo Preparation (Prepare Once)

Prepare a placebo in the same way as the Sample Preparation but using placebo tablets.

### Sample Preparation

Transfer 20 pre-weighed tablets into a 500 ml amber volumetric flask.

Add approximately 300 ml of Mobile Phase. Sonicate and shake mechanically until the tablets have fully disintegrated. Allow to equilibrate to room temperature. Dilute to volume with Mobile Phase. Mix well. Centrifuge the solution or alternatively filter through a 0.45 μm RC filter. Transfer 5.0 ml of the solution into a 20 ml amber volumetric flask, dilute with mobile phase to volume and mix.

### System Suitability Criteria

| Parameter | Specification |
|---|---|
| Tailing factor (Calculate for first | ≤2.0 |
| reference standard injection) | |
| Signal to noise | ≥10:1 |
| Standard Verification | 95.0%-105.0% |
| % RSD of peak area responses for | ≤5.0% |
| the 5 reference standard A injections | |
| % RSD for peak area responses for | ≤5.0% |
| bracketing standard injections | |
| throughout run | |
| % RSD for retention times for the 5 | ≤1.0% |
| reference standard A injections | |

### Calculation

$$\% Impurity = \frac{PA ( spl )}{PA ( std )} \times \frac{Std Wgt ( mg )}{100 ( mL )} \times \frac{2 ( mL )}{200 ( mL )} \times \frac{500 ( mL )}{Spl Wgt} \times \frac{20 ( mL )}{5 ( mL )} \times \frac{ACW}{LC} \times \frac{P}{100} \times \frac{136.19}{234.28} \times 100$$

where:

| | |
|---|---|
| PA(spl): | Peak area of Impurity in the Sample Chromatogram. |
| PA(std): | Mean Peak area of Phenelzine Sulphate in the |
| | Bracketing Standard Chromatograms. |
| Std Wgt | Standard weight of Phenelzine Sulphate Reference |
| | Standard (mg). |
| Spl Wgt: | Sample weight (mg). |
| ACW: | Average Tablet Weight. |
| LC: | 15 mg |
| P: | Potency of Phenelzine Sulphate |

### 1.10 Dissolution

Use the USP 1 dissolution apparatus. Fill the vessels with 900 ml of purified water and heat to 37±0.5° C. Place one tablet into each of six baskets, lower the baskets into the medium and rotate at 150 rpm. Remove a 5 ml sample from each vessel at 45 minutes. Pass each sample through a single use 0.45 μm RC filter. Alternatively, centrifuge the sample. Analyse the samples using the HPLC method described below:

### Standard Solution

Accurately weigh approximately 30.00 mg of phenelzine sulfate standard into a 100 ml volumetric flask. Dissolve in approximately 80 ml of mobile phase by sonicating for 10 minutes, make to volume with diluent and shake thoroughly. Dilute 5 ml of this solution to 50 ml with mobile phase.

### Sample Solution

Directly inject the samples removed from the dissolution vessel.

### Procedure

Samples are analysed using the HPLC procedure as follows.

### Reagents

Purified water
Methanol
Sodium 1-octanesulfonate
Monobasic potassium phosphate
Orthophosphoric acid 85%

Ion-pair solution: Dissolve 6.8 g of monobasic potassium phosphate and 2.16 g of sodium 1-octanesulfonate in 1000 ml of water. Adjust to pH 3.0±0.1 with orthophosphoric acid. (Quantities proportionally larger or smaller than specified here may be used).

### HPLC Conditions

| | |
|---|---|
| Column: | Symmetry C18 5 μm, |
| | 150 mm × 3.9 mm, or equivalent |
| Guard Column: | Security Guard C18, 3 × 4 mm |
| | (Phenomenex or equivalent) |
| Mobile phase: | Ion-pair solution : Methanol (60:40 v/v) |
| Flow rate: | 1.0 ml / minute |
| Column temperature: | 20° C. |
| Detection: | UV at 210 nm |
| Injection: | 20 μl |

### System Suitability Criteria

| Parameter | Specification |
|---|---|
| Tailing factor (Calculate for first | ≤2.0 |
| reference standard injection) | |
| Standard Verification | 95.0%-105.0% |
| % RSD of peak area responses for | ≤5.0% |
| the 5 reference standard A injections | |
| % RSD for peak area responses for | ≤5.0% |
| bracketing standard injections | |
| throughoutrun | |
| % RSD for retention times for the 5 | ≤1.0% |
| reference standard A injections | |

### Calculation

$$\% Dissolved = \frac{PA ( spl )}{PA ( std )} \times \frac{Std Wgt ( mg )}{100 ( mL )} \times \frac{5 ( mL )}{50 ( mL )} \times \frac{900 ( mL )}{LC} \times \frac{P}{100} \times \frac{136.19}{234.28} \times 100$$

where:

| | |
|---|---|
| PA(spl): | Peak area of Phenelzine in the Sample Chromatogram. |
| PA(std): | Mean Peak area of Phenelzine in the Bracketing |
| | Standard Chromatograms. |
| Std wgt(mg): | Standard weight of Phenelzine Sulphate Reference |
| | Standard (mg). |
| LC: | 15 mg Tablets. |
| P: | Potency of Phenelzine Sulphate Reference. |

### 1.11 Microbial Purity

Perform Microbial Enumeration Tests for Total Aerobic Microbial Count (TAMC) and Total Yeast and Mould Count (TYMC) as per Ph. Eur. 2.6.12 using the plate count method.

Sample dilutions 1:10.

Perform test for Specified Microorganisms, E. coli as per Ph. Eur. 2.6.13.

### Results

The results of the drug product stability analysis are shown in Tables 3 to 10 where it can be seen that all parameters met the specification limits for storage at 5° C.±3° C. and at 25±2° C./60±5% RH. These data support the registered shelf life of 24 months for Phenelzine film coated tablets when stored between 2° C. to 8° C. in a refrigerator, in its original packaging.

TABLE 3
Batch 533343 at 5° C. - INVERTED

| Test | Specification | Initial | 3 months | 6 months | 12 months | 18 months | 24 months |
|---|---|---|---|---|---|---|---|
| Description | Smooth, uniformly coated, | Complies | Complies | Complies | Complies | Complies | Complies |
| | biconvex tablets with | | | | | | |
| | rounded edges, and having | | | | | | |
| | not more thana slight odour | | | | | | |
| Colour | Orange and free from mottling | Complies | Complies | Complies | Complies | Complies | Complies |
| Coating | Free from defects such as | Complies | Complies | Complies | Complies | Complies | Complies |
| | chips, spots, pits, cracks etc. | | | | | | |
| Loss on drying | Max. 1.5% w/w | 0.8% w/w | 0.8% w/w | 0.6% w/w | 0.9% w/w | 1.2% w/w³ | 0.9% w/w |
| Assay - phenelzine | 13.5 to 16.5 mg per | 15.0 mg/ | 14.9 mg/ | 15.0 mg/ | 14.9 mg/ | 15.0 mg/ | 14.8 mg/ |
| | averagetablet | tab | tab | tab | tab | tab | tab |
| Identification | Phenelzine retention | Complies | | | | Complies | Complies |
| | timecomplies | | | | | | |
| | Complies with BP | Complies | | | | Complies | Complies |
| | Identificationtest B | | | | | | |
| Related substances | Individual impurity NMT 0.5% | <0.1%¹ | 0.14% | 0.10% | <0.10%² | <0.10% | 0.20% |
| | Total impurities NMT 1.5% | <0.1%¹ | 0.14% | 0.10% | <0.10%² | <0.10% | 0.20% |
| Uniformity of Dosage | Level 1 (N = 10): AV ≤ | AV = 2.2 | | | | AV = 3.2 | AV = 2.2 |
| Units(Ph. Eur. 2.9.40) | 15.0Level 2 (N = 30): AV ≤ | Mean: | | | | Mean: | Mean: |
| | 15.0 | 101.3% LC | | | | 102.1% LC | 99.9% LC |
| | No individual less than 0.75M | Range: | | | | Range: | Range: |
| | orgreater than 1.25M. | 100.1- | | | | 100.4- | 98.0- |
| | | 103.2% LC | | | | 103.7% LC | 101.6% LC |
| Dissolution test | Complies with the BP. | 99% | 102% | 103% | 101% | 101% | 100% |
| | Not less than 80% released | (95-103%) | (100-103%) | (101-105%) | (98-103%) | (98-102%) | (98-104%) |
| | after45 minutes (Q = 75%) | | | | | | |
| Condition of package | No defects | Complies | Complies | Complies | Complies | Complies | Complies |
| Microbial purity | TAMC: NMT 2000 CFU/g | N/P | | | | Complies | Complies |
| | TYMC: NMT 200 CFU/g | N/P | | | | Complies | Complies |
| | Absence of E. coli in 1 g | N/P | | | | Complies | Complies |

¹Results reported to one decimal place at initial.
²Not detected.
³Mean taken from Section A & B results.
N/P—Not Performed: microbial purity test performed 1 in 10 batches.

TABLE 4
Batch 533343 at 5° C. - UPRIGHT

| Test | Specification | Initial | 3 months | 6 months | 12 months | 18 months | 24 months |
|---|---|---|---|---|---|---|---|
| Description | Smooth, uniformly coated, | Complies | Complies | Complies | Complies | Complies | Complies |
| | biconvex tablets with | | | | | | |
| | rounded edges, and having | | | | | | |
| | not more thana slight odour | | | | | | |
| Colour | Orange and free from mottling | Complies | Complies | Complies | Complies | Complies | Complies |
| Coating | Free from defects such as | Complies | Complies | Complies | Complies | Complies | Complies |
| | chips, spots, pits, cracks etc. | | | | | | |
| Loss on drying | Max. 1.5% w/w | 0.8% w/w | 0.7% w/w | 0.5% w/w | 0.9% w/w | 1.2% w/w | 0.9% w/w |
| Assay - phenelzine | 13.5 to 16.5 mg per | 15.0 mg/ | 14.9 mg/ | 15.0 mg/ | 14.9 mg/ | 14.9 mg/ | 14.8 mg/ |
| | averagetablet | tab | tab | tab | tab | tab | tab |
| Identification | Phenelzine retention | Complies | | | | Complies | Complies |
| | timecomplies | | | | | | |
| | Complies with BP | Complies | | | | Complies | Complies |
| | Identificationtest B | | | | | | |
| Related substances | Individual impurity NMT 0.5% | <0.1%¹ | 0.13% | 0.11% | 0.11% | <0.10% | 0.16% |
| | Total impurities NMT 1.5% | <0.1%¹ | 0.13% | 0.11% | 0.11% | <0.10% | 0.16% |
| Uniformity of Dosage | Level 1 (N = 10): AV ≤ | AV = 2.2 | | | | AV = 1.9 | AV = 3.6 |
| Units(Ph. Eur. 2.9.40) | 15.0Level 2 (N = 30): AV ≤ | Mean: | | | | Mean: | Mean: |
| | 15.0 | 101.3% LC | | | | 100.9% LC | 99.3% LC |
| | No individual less than 0.75M | Range: | | | | Range: | Range: |
| | orgreater than 1.25M. | 100.1- | | | | 99.9- | 96.8- |
| | | 103.2% LC | | | | 101.8% LC | 102.1% LC |
| Dissolution test | Complies with the BP. | 99% | 101% | 101% | 102% | 100% | 100% |
| | Not less than 80% released | (95-103%) | (100-105%) | (100-102%) | (100-105%) | (97-104%) | (99-101%) |
| | after45 minutes (Q = 75%) | | | | | | |
| Condition of package | No defects | Complies | Complies | Complies | Complies | Complies | Complies |
| Microbial purity | TAMC: NMT 2000 CFU/g | N/P | | | | Complies | Complies |
| | TYMC: NMT 200 CFU/g | N/P | | | | Complies | Complies |
| | Absence of E. coli in 1 g | N/P | | | | Complies | Complies |

¹Results reported to one decimal place at initial.
N/P—Not Performed: microbial purity test performed 1 in 10 batches.

TABLE 5
Batch 533343 at 25°C/60% RH-INVERTED

| Test | Specificatio | Initial | 1 month | 3 months | 6 months |
|---|---|---|---|---|---|
| Description | Smooth, uniformly coated, | Complies | Complies | Complies | Complies |
| | biconvex tablets with rounded | | | | |
| | edges, and havingnot more than a [symbol] | | | | |
| Colour | Orange and free from mottling | Complies | Complies | Complies | Complies |
| Coating | Free from defects such as chips, | Complies | Complies | Complies | Complies |
| | spots. pits. cracks etc. | | | | |
| Loss on drying | Max. 1.5% w/W | 0.8% w/w | 0.2% w/w | 0.9% w/w | 0.7% w/W |
| Assay-phenelzine | 13.5 to 16.5 mg per average tablet | 15.0 mg/tab | 14.8 mg/tab | 14.3 mg/tab | 14.0 mg/tab |
| Identification | Phenelzine retention time complies | Complies | | | |
| | Complies with BP Identification test B | Complies | | | |
| Related substances | Individual impurity NMT 0.5% | <0.1%¹ | 0.20% | 0.25% | 0.37% |
| | Total impurities NMT 1.5% | <0.1%¹ | 0.33% | 0.46% | 0.75% |
| Uniformitv of | Level 1 (N = 10): AV ≤ 15.0 | AV = 2.2 | | | AV = 7.0 |
| Dosage Units | Level 2 (N = 30): AV ≤ 15.0 | Mean: 101.3% LC | | | Mean: 94.0% LC |
| (Ph. Eur. 2.9.40) | No individual less than 0.75 M or | Range: | | | Range: |
| | than 1.25 M. | 100.1- [symbol] | | | 92.2-95.2% LC |
| Dissolution test | Complies with the BP. | 99% | 99% | 96% | 94% (93-98%) |
| | Not less than 80% released | (95-103%) | (95-101%) | (94-98%) | |
| | [symbol] | | | | |
| Condition of package | No defects | Complies | Complies | Complies | Complies |
| Microbial purity | TAMC: NMT 2000 CFU/g | N/P | | | |
| | TYMC: NMT 200 CFU/g | N/P | | | |
| | Absence of E.coli in 1 g | N/F | | | |

¹Results reported to one decimal place at initial.
N/P-Not Performed: microbial purity test performed 1 in 10 batches. [symbol]
indicates data missing or illegible when filed

TABLE 6
Batch 533343 at 25°C/60% RH-UPRIGHT

| Test | Specificatio | Initial | 1 month | 3 months | 6 months |
|---|---|---|---|---|---|
| Description | Smooth, uniformly coated, | Complies | Complies | Complies | Complies |
| | biconvex tablets with rounded | | | | |
| Colour | edges, and havingnot more than a [symbol] | Complies | Complies | Complies | Complies |
| | Orange and free from mottling | | | | |
| Coating | Free from defects such as chips, | Complies | Complies | Complies | Complies |
| | spots. pits. cracks etc. | | | | |
| Loss on drying | Max. 1.5% w/w | 0.8% w/w | 0.2% w/w | 0.8% w/w | 0.1% w/w |
| Assay-phenelzine | 13.5 to 16.5 mg per average tablet | 15.0 mg/tab | 14.8 mg/tab | 14.3 mg/tab | 14.0 mg/tab |
| Identification | Phenelzine retention time complies | Complies | | | |
| | Complies with BP Identification test B | Complies | | | |
| Related substances | Individual impurity NMT 0.5% | <0.1%¹ | 0.20% | 0.22% | 0.36% |
| | Total impurities NMT 1.5% | <0.1%¹ | 0.32% | 0.40% | 0.74% |
| Uniformity of | Level 1 (N = 10): AV ≤ 15.0 | AV = 2.2 | | | AV = 7.0 |
| Dosage Units | Level 2 (N = 30): AV ≤ 15.0 | Mean: 101.3% LC | | | Mean: 94.1% LC |
| (Ph. Eur. 2.9.40) | No individual less than 0.75 M or | Range: | | | Range: |
| | than 1.25 M. | 100.1- [symbol] | | | 92.5-95.4% LC |
| Dissolution test | Complies with the BP. | 99% | 98% | 98% | 95% |
| | Not less than 80% released | (95-103%) | (97-99%) | (97-100%) | (92-96%) |
| | [symbol] | | | | |
| Condition of package | No defects | Complies | Complies | Complies | Complies |
| Microbial purity | TAMC: NMT 2000 CFU/g | N/P | | | |
| | TYMC: NMT 200 CFU/g | N/P | | | |
| | Absence of E.coli in 1 g | N/P | | | |

¹Results reported to one decimal place at initial.
N/P-Not Performed: microbial purity test performed 1 in 10 batches. [symbol]
indicates data missing or illegible when filed

TABLE 7
Batch 533474 at 5° C. - INVERTED

| Test | Specification | Initial | 3 months | 6 months | 12 months | 18 months | 24 months |
|---|---|---|---|---|---|---|---|
| Description | Smooth, uniformly coated, | Complies | Complies | Complies | Complies | Complies | Complies |
| | biconvex tablets with | | | | | | |
| | rounded edges, and having | | | | | | |
| | not more thana slight odour | | | | | | |
| Colour | Orange and free from mottling | Complies | Complies | Complies | Complies | Complies | Complies |
| Coating | Free from defects such as | Complies | Complies | Complies | Complies | Complies | Complies |
| | chips, spots, pits, cracks etc. | | | | | | |
| Loss on drying | Max. 1.5% w/w | 1.0% w/w | 0.9% w/w | 0.6% w/w | 0.8% w/w | 1.1% w/w | 0.9% w/w |
| Assay - phenelzine | 13.5 to 16.5 mg per | 15.1 mg/ | 15.0 mg/ | 15.2 mg/ | 15.0 mg/ | 15.1 mg/ | 15.0 mg/ |
| | averagetablet | tab | tab | tab | tab | tab | tab |
| Identification | Phenelzine retention | Complies | | | | Complies | Complies |
| | timecomplies | | | | | | |
| | Complies with BP | Complies | | | | Complies | Complies |
| | Identificationtest B | | | | | | |
| Related substances | Individual impurity NMT 0.5% | <0.1%¹ | 0.12% | 0.13% | 0.11% | <0.10% | 0.21% |
| | Total impurities NMT 1.5% | <0.1%¹ | 0.12% | 0.13% | 0.11% | <0.10% | 0.32% |
| Uniformity of Dosage | Level 1 (N = 10): AV ≤ | AV = 2.7 | | | | AV = 3.8 | AV = 2.2 |
| Units(Ph. Eur. 2.9.40) | 15.0Level 2 (N = 30): AV ≤ | Mean: | | | | Mean: | Mean: |
| | 15.0 | 102.1% LC | | | | 99.9% LC | 100.2% LC |
| | No individual less than 0.75M | Range: | | | | Range: | Range: |
| | orgreater than 1.25M. | 100.1- | | | | 96.5- | 99.1- |
| | | 103.0% LC | | | | 101.9% LC | 101.3% LC |
| Dissolution test | Complies with the BP. | 96% | 102% | 102% | 102% | 101% | 100% |
| | Not less than 80% released | (89-104%) | (100-103%) | (101-103%) | (101-104%) | (98-103%) | (99-102%) |
| | after45 minutes (Q = 75%) | | | | | | |
| Condition of package | No defects | Complies | Complies | Complies | Complies | Complies | Complies |
| Microbial purity | TAMC: NMT 2000 CFU/g | Complies | | | | Complies | Complies |
| | TYMC: NMT 200 CFU/g | Complies | | | | Complies | Complies |
| | Absence of E. coli in 1 g | Complies | | | | Complies | Complies |

¹Results reported to one decimal place at initial.

TABLE 8
Batch 533474 at 5° C. - UPRIGHT

| Test | Specification | Initial | 3 months | 6 months | 12 months | 18 months | 24 months |
|---|---|---|---|---|---|---|---|
| Description | Smooth, uniformly coated, | Complies | Complies | Complies | Complies | Complies | Complies |
| | biconvex tablets with | | | | | | |
| | rounded edges, and having | | | | | | |
| | not more thana slight odour | | | | | | |
| Colour | Orange and free from mottling | Complies | Complies | Complies | Complies | Complies | Complies |
| Coating | Free from defects such as | Complies | Complies | Complies | Complies | Complies | Complies |
| | chips, spots, pits, cracks etc. | | | | | | |
| Loss on drying | Max. 1.5% w/w | 1.0% w/w | 0.9% w/w | 0.5% w/w | 0.8% w/w | 1.0% w/w | 0.9% w/w |
| Assay - phenelzine | 13.5 to 16.5 mg per | 15.1 mg/ | 15.0 mg/ | 15.2 mg/ | 14.9 mg/ | 15.2 mg/ | 14.9 mg/ |
| | averagetablet | tab | tab | tab | tab | tab | tab |
| Identification | Phenelzine retention | Complies | | | | Complies | Complies |
| | timecomplies | | | | | | |
| | Complies with BP | Complies | | | | Complies | Complies |
| | Identificationtest B | | | | | | |
| Related substances | Individual impurity NMT 0.5% | <0.1%¹ | 0.13% | <0.1%¹ | 0.10% | <0.10% | 0.18% |
| | Total impurities NMT 1.5% | <0.1%¹ | 0.13% | <0.1%¹ | 0.10% | <0.10% | 0.29% |
| Uniformity of Dosage | Level 1 (N = 10): AV ≤ | AV = 2.7 | | | | AV = 1.4 | AV = 1.9 |
| Units(Ph. Eur. 2.9.40) | 15.0Level 2 (N = 30): AV ≤ | Mean: | | | | Mean: | Mean: |
| | 15.0 | 102.1% LC | | | | 100.5% LC | 100.5% LC |
| | No individual less than 0.75M | Range: | | | | Range: | Range: |
| | orgreater than 1.25M. | 100.1- | | | | 99.5- | 98.7- |
| | | 103.0% LC | | | | 101.2% LC | 101.5% LC |
| Dissolution test | Complies with the BP. | 96% | 99% | 102% | 102% | 103% | 101% |
| | Not less than 80% released | (89-104%) | (98-101%) | (102-103%) | (96-105%) | (101-105%) | (100-102%) |
| | after45 minutes (Q = 75%) | | | | | | |
| Condition of package | No defects | Complies | Complies | Complies | Complies | Complies | Complies |
| Microbial purity | TAMC: NMT 2000 CFU/g | Complies | | | | Complies | Complies |
| | TYMC: NMT 200 CFU/g | Complies | | | | Complies | Complies |
| | Absence of E. coli in 1 g | Complies | | | | Complies | Complies |

¹Results reported to one decimal place at initial.

TABLE 9
Batch 533474 at 25° C./60% RH-INVERTED

| Test | Specificatio | Initial | 1 month | 3 months | 6 months |
|---|---|---|---|---|---|
| Description | Smooth, uniformly coated, | Complies | Complies | Complies | Complies |
| | biconvex tablets with rounded | | | | |
| Colour | edges. and havingnot more than a [symbol] | Complies | Complies | Complies | Complies |
| | Orange and free from mottling | | | | |
| Coating | Free from defects such as chips, | Complies | Complies | Complies | Complies |
| | spots. pits. cracks etc. | | | | |
| Loss on drying | Max. 1.5% w/w | 1.0% w/w | 0.7% w/w | 0.9% ww | 0.7% w/w |
| Assay-phenelzine | 13.5 to 16.5 mg per average tablet | 15.1 mg/tab | 14.9 mg/tab | 14.6 mg/tab | 14.2 mg/tab |
| Identification | Phenelzine retention time complies | Complies | | | |
| | Complies with BP Identification test B | Complies | | | |
| Related substances | Individual impurity NMT 0.5% | <0.1%¹ | 0.32% | 0.21% | 0.42% |
| | Total impurities NMT 1.5% | <0.1%¹ | 0.90% | 0.41% | 0.83% |
| Uniformity of | Level 1 (N = 10): AV ≤ 15.0 | AV = 2.7 | | | AV = 6.1 |
| Dosage Units | Level 2 (N = 30): AV ≤ 15.0 | Mean: 102.1% LC | | | Mean: 5.5% LC |
| (Ph. Eur. 2.9.40) | No individual less than 0.75 M or | Range: | | | Range: |
| | than 1.25 M. | 100.1-103.0% [symbol] | | | 94.1-96.6% LC |
| Dissolution test | Complies with the BP. | 96% | 99% | 98% | 97% |
| | Not less than 80% released | (89-104%) | (97-100%) | (96-99%) | (95-99%) |
| | [symbol] | | | | |
| Condition of package | No defects | Complies | Complies | Complies | Complies |
| Microbial purity | TAMC: NMT 2000 CFU/g | Complies | | | |
| | TYMC: NMT 200 CFU/g | Complies | | | |
| | Absence of E.coli in 1g | Complies | | | |

¹Results reported to one decimal place at initial. [symbol]
indicates data missing or illegible when filed

TABLE 10
Batch 533474 at 25° C./60% RH-UPRIGHT

| Test | Specificatio | Initial | 1 month | 3 months | 6 months |
|---|---|---|---|---|---|
| Description | Smooth, uniformly coated, | Complies | Complies | Complies | Complies |
| | biconvex tablets with rounded | | | | |
| Colour | edges. and havingnot more than a [symbol] | Complies | Complies | Complies | Complies |
| | Orange and free from mottling | | | | |
| Coating | Free from defects such as chips, | Complies | Complies | Complies | Complies |
| | spots. pits. cracks etc. | | | | |
| Loss on drying | Max. 1.5% w/w | 1.0% w/w | 0.4% w/w | 0.9% w/w | 0.5% w/w |
| Assay-phenelzine | 13.5 to 16.5 mg per average tablet | 15.1 mg/tab | 15.0 mg/tab | 14.5 mg/tab | 14.2 mg/tab |
| Identification | Phenelzine retention time complies | Complies | | | |
| | Complies with BP Identification test B | Complies | | | |
| Related substances | Individual impurity NMT 0.5% | <0.1%¹ | 0.26% | 0.22% | 0.39% |
| | Total impurities NMT 1.5% | <0.1%¹ | 0.57% | 0.42% | 0.78% |
| Uniformity of | Level 1 (N = 10): AV ≤ 15.0 | AV = 2.7 | | | AV = 5.0 |
| Dosage Units | Level 2 (N = 30): AV ≤ 15.0 | | | | |
| (Ph. Eur. 2.9.40) | No individual less than 0.75 M or | Mean: 102.1% LC | | | Mean: 95.4% LC |
| | than 1.25 M. | Range: | | | Range: |
| | | 100.1- [symbol] | | | 93.9-96.5% LC |
| Dissolution test | Complies with the BP. | 96% | 99% | 101% | 96% |
| | Not less than 80% released | (89-104%) | (98-100%) | (93-106%) | (95-97%) |
| | [symbol] | | | | |
| Condition of package | No defects | Complies | Complies | Complies | Complies |
| Microbial purity | TAMC: NMT 2000 CFU/g | Complies | | | |
| | TYMC: NMT 200 CFU/g | Complies | | | |
| | Absence of E.coli in 1g | Complies | | | |

¹Results reported to one decimal place at initial. [symbol]
indicates data missing or illegible when filed

### Example 2: In Use Stability Analysis

### 1. Objective

The purpose of this report is to provide data that supports the in-use stability of Phenelzine 15 mg tablets to support the overall stability study of Phenelzine.

### 2. Background

In-use stability is assessed by simulating the use of a multi-dose container by the patient, performing analytical testing on the samples before and after storage of the container incorporating regular removal of tablets and trending of the results to ensure they meet specification over the course of the in-use study. The study is used to determine the impact of the in-use conditions on product quality of a batch at the beginning of its anticipated shelf-life.

A 24 Hour Temperature Excursion Study was also performed on samples stored (upright & inverted) at 5° C.±3° C. to assess product quality.

The duration of the in-use stability studies has been conducted in accordance with the details registered in the current Summary of Product Characteristics (SmPC) and include 200 Day, 34 Day and 17 Day studies as well as a 24 Hour Temperature Excursion Study.

The sample cartons and bottles were opened and tablets removed.

### 3. Materials and Equipment Used

| Material | Manufacturer | Lot Number | Expiry Date |
|---|---|---|---|
| Purified Water | In House | N/A | 8 Jul. 2022 |
| | | | 9 Jul. 2022 |
| | | | 15 Jul. 2022 |
| | | | 16 Jul. 2022 |
| | | | 17 Jul. 2022 |
| | | | 26 Aug. 2022 |
| | | | 27 Aug. 2022 |
| | | | 21 Oct. 2022 |
| | | | 22 Oct. 2022 |
| | | | 9 Dec. 2022 |
| | | | 12 Dec. 2022 |
| | | | 13 Dec. 2022 |
| | | | 14 Dec. 2022 |
| Methanol | Fisher | 2215779 | 12 Jul. 2025 |
| | | 2224894 | 20 Sep. 2025 |
| | | 2193682 | 4 Jul. 2025 |
| | | 2213870 | 5 Jul. 2025 |
| | | 2224894 | 5 Jul. 2025 |
| | | 2228876 | 22 Aug. 2025/ |
| | | 2211493 | 17 Oct. 2025 |
| | | 2220149 | 14 Oct. 2025 |
| | | 2233661 | 9 Dec. 2025 |
| | | 2236457 | 5 Dec. 2025 |
| | | 2231171 | 8 Dec. 2025 |
| | | | 8 Dec. 2025 |
| Helium | BOC | N/A | 12 Apr. 2023 |
| Orthophosphoric | Fisher | 1916991 | 10 Dec. 2022/ |
| Acid | | 2052789 | 16 Jul. 2023 |
| | | | 28 Oct. 2023 |
| 0.45 μm Nylon | Whatman | A29841522 | 30 Jun. 2025/ |
| Filters | | A29484468 | 1 Jul. 2025 |
| | | A29228333 | 30 Jan. 2024 |
| | | | Apr. 2023 |
| Potassium | Acros | A0426630 | 28 Feb. 2024 |
| Phosphate | Organics | | |
| Monobasic | | | |
| Sodium 1- | Thermo- | 10232675 | |
| octanesulfanate | scientific | | 1 Apr. 2026 |
| Monobasic | Fisher | 1848174 | 15 Jun. 2027 |
| Potassium | | 2174660 | 6 Dec. 2026 |
| Phosphate | | | |
| Chromafil PVDF | Macherey | 7.074 | N/A |
| Filter 0.45 μm | Nagal | | |
| Minisart RC | Sartorius | 217573103 | N/A N/A |
| 0.45 μm Filters | | 211573103 | |

| | Instrument | |
|---|---|---|
| Equipment | Number | Calibration Due Date |
| Elga | RDA-1270 | 16 Sep. 2022/14 Mar. 2023 |
| | E0202 | 6 Oct. 2022/3 Apr. 2023 |
| | RDA-1271 | 16 Sep. 2022 |
| Balance | RDA-1039 | 15 Jul. 2022/19 Aug. 2022/12 Nov. 2022/ |
| | | 4 Dec. 2022/9 Dec. 2022 |
| | E0615 | 22 Aug. 2022/27 Oct. 2022/2 Jan. 2023 |
| | E0427 | 23 Jul. 2022/06 Nov. 2022/11 Dec. 2022 |
| | RDA-1236 | 13 Jul. 2022 |
| | E0108 | 13 Nov. 2022/18 Dec. 2022 |
| Stirring plate | RDA-1246 | N/A |
| | RDA-1245 | N/A |
| | RDA-1244 | N/A |
| | RDA-1024 | N/A |
| Vacuum | RDA-1062 | N/A |
| Pump | E0362 | N/A |
| Sonicator | RDA-1278 | N/A |
| | RDA-1027 | N/A |
| | E0864 | N/A |
| Orbital | RDA-1276 | N/A |
| Shaker | RDA-1242 | N/A |
| | RDA-1058 | N/A |
| | E0180 | N/A |
| PH Meter | RDA-1237 | 2 Dec. 2022 |
| | E1450 | 5 Oct. 2022 |
| HPLC | RDA-1225 | 8 Dec. 2022 |
| | RDA-1228 | 8 Dec. 2022 |
| | RDA-1230 | 9 Dec. 2022 |
| | RDA-1231 | 9 Dec. 2022 |
| | RDA-1201 | 2 Dec. 2022 |
| | RDA-1229 | 9 Dec. 2022 |
| | E0695 | 31 Aug. 2023 |
| | E0696 | 28 Sep. 2023 |
| Centrifuge | E0777 | 12 Apr. 2023 |
| Timer | E1509 | 12 Apr. 2023 |
| | E1367 | 12 Apr. 2023 |
| | RDA-1077 | 13 Apr. 2023 |
| | E0593 | 10 Mar. 2023 |
| | RDA-1078 | 13 Apr. 2023 |
| | E0598 | Jan. 2023 |
| Dissolution | E0035 | 19 Jan. 2023 |
| Bath | E0325 | 19 Jan. 2023 |
| | RDA-1107 | 12 Oct. 2022/4 Apr. 2023 |
| | RDA-1075 | 13 Oct. 2022/5 Apr. 2023 |
| | E0206 | 18 Jun. 2023 |
| | E0412 | 14 Dec. 2022 |
| Calibrated | E1453 | 28 Sep. 2022 |
| Ruler | E0049 | 27 Jul. 2022/07 Jul. 2023 |

### 4. Results

Phenelzine 15 mg Bulk Tablets batch CM1090PR was evaluated initially and packed sample cartons and bottles for all in-use stability studies (upright & inverted) were placed in the laboratory fridge at 5° C.±3° C. All samples were analysed for Appearance, Identification HPLC, Assay by HPLC, Related Substances, Dissolution by HPLC and Microbial Purity at 17 days (17 Day Study), 34 days (34 Day Study), 200 days (200 Day Study) and 49 days (24 Hour Temperature Excursion Study).

All results are outlined in the stability summary sheet listed in Section 5 of this report.

### 4.1 Appearance Results 17 Day Study 1

The Appearance test results for all upright and inverted samples pass the specification at the initial time point and over 17 days on stability at 5° C.±3° for CM1090PR.

### 34 Day Study 2

The Appearance test results for all upright and inverted samples pass the specification at the initial time point and over 34 days on stability at 5° C.±3° for CM1090PR.

### 200 Day Study 3

The Appearance test results for all upright and inverted samples pass the specification at the initial time point and over 200 days on stability at 5° C.±3° for CM1090PR.

### 24 Hour Temperature Excursion Study 4

The Appearance test results for all upright and inverted samples pass the specification at the initial time point and over 49 days on stability at 5° C.±3° for CM1090PR.

### 4.2 Identification HPLC Results

### 17 Day Study 1

The Identification HPLC test results for all upright and inverted samples pass the specification at the initial time point and over 17 days on stability at 5° C.±3° for CM1090PR.

### 34 Day Study 2

The Identification HPLC test results for all upright and inverted samples pass the specification at the initial time point and over 34 days on stability at 5° C.±3° for CM1090PR.

### 200 Day Study 3

The Identification HPLC test results for all upright and inverted samples pass the specification at the initial time point and over 200 days on stability at 5° C.±3° for CM1090PR.

### 24 Hour Temperature Excursion Study 4

The Identification HPLC test results for all upright and inverted samples pass the specification at the initial time point and over 49 days on stability at 5° C.±3° for CM1090PR.

### 4.3 Assay by HPLC Results

### 17 Day Study 1

The Assay by HPLC test results for all upright and inverted samples pass the specification at the initial time point and over 17 days on stability at 5° C.±3° for CM1090PR.

### 34 Day Study 2

The Assay by HPLC test results for all upright and inverted samples pass the specification at the initial time point and over 34 days on stability at 5° C.±3° for CM1090PR.

### 200 Day Study 3

The Assay by HPLC test results for all upright and inverted samples pass the specification at the initial time point and over 200 days on stability at 5° C.±3° for CM1090PR.

24 Hour Temperature Excursion Study 4 The Assay by HPLC test results for all upright and inverted samples pass the specification at the initial time point and over 49 days on stability at 5° C.±3° for CM1090PR.

### 4.4 Related Substances Results

### 17 Day Study 1

The Related Substances test results for all upright and inverted samples pass the specification at the initial time point and over 17 days on stability at 5° C.±3° for CM1090PR.

### 34 Day Study 2

The Related Substances test results for all upright and inverted samples pass the specification at the initial time point and over 34 days on stability at 5° C.±3° for CM1090PR.

### 200 Day Study 3

The Related Substances test results for all upright and inverted samples pass the specification at the initial time point and over 200 days on stability at 5° C.±3° for CM1090PR.

### 24 Hour Temperature Excursion Study 4

The Related Substances test results for all upright and inverted samples pass the specification at the initial time point and over 49 days on stability at 5° C.±3° for CM1090PR.

### 4.5 Dissolution by HPLC Results

### 17 Day Study 1

The Dissolution by HPLC test results for all upright and inverted samples pass the specification at the initial time point and over 17 days on stability at 5° C.±3° for CM1090PR.

### 34 Day Study 2

The Dissolution by HPLC test results for all upright and inverted samples pass the specification at the initial time point and over 34 days on stability at 5° C.±3° for CM1090PR.

### 200 Day Study 3

The Dissolution by HPLC test results for all upright and inverted samples pass the specification at the initial time point and over 200 days on stability at 5° C.±3° for CM1090PR.

### 24 Hour Temperature Excursion Study 4

The Dissolution by HPLC test results for all upright and inverted samples pass the specification at the initial time point and over 49 days on stability at 5° C.±3° for CM1090PR.

### 4.6 Microbial Purity Results

### 17 Day Study 1

The Microbial Purity test results for all upright and inverted samples pass the specification at the initial time point and over 17 days on stability at 5° C.±3° for CM1090PR.

### 34 Day Study 2

The Microbial Purity test results for all upright and inverted samples pass the specification at the initial time point and over 34 days on stability at 5° C.±3° for CM1090PR.

### 200 Day Study 3

The Microbial Purity test results for all upright and inverted samples pass the specification at the initial time point and over 200 days on stability at 5° C.±3° for CM1090PR.

### 24 Hour Temperature Excursion Study 4

The Microbial Purity test results for all upright and inverted samples pass the specification at the initial time point and over 49 days on stability at 5° C.±3° for CM1090PR.

### 5. Summary of Results

The following batch of Phenelzine 15 mg Tablets was manufactured and released:
CM1090PR

The results of this analysis are summarised in Table 11. In general, Example 2 shows that the product is stable during in-use for up to 200 days when stored between 2° C. to 8° C. in a refrigerator, in its original packaging.

TABLE 11
Summary of Results of Drug Product Stability Analysis
Assay (mg per average tablet)

| Time Point (Days) | 0 | 17 | 34 | 49 | 50 | 100 | 150 | 200 |
|---|---|---|---|---|---|---|---|---|
| Upper Spec | 16.5 | 16.5 | 16.5 | 16.5 | 16.5 | 16.5 | 16.5 | 16.5 |
| Lower Spec | 13.5 | 13.5 | 13.5 | 13.5 | 13.5 | 13.5 | 13.5 | 13.5 |

Individual Impurity

| Time Point (Days) | 0 | 17 | 34 | 49 | 50 | 100 | 150 | 200 |
|---|---|---|---|---|---|---|---|---|
| Upper Spec | 0.5% | 0.5% | 0.5% | 0.5% | 0.5% | 0.5% | 0.5% | 0.5% |

Total Impurity

| Time Point (Days) | 0 | 17 | 34 | 49 | 50 | 100 | 150 | 200 |
|---|---|---|---|---|---|---|---|---|
| Upper Spec | 1.5% | 1.5% | 1.5% | 1.5% | 1.5% | 1.5% | 1.5% | 1.5% |

Dissolution

| Time Point (Days) | 0 | 17 | 34 | 49 | 50 | 100 | 150 | 200 |
|---|---|---|---|---|---|---|---|---|
| Upper Spec | 80% | 80% | 80% | 80% | 80% | 80% | 80% | 80% |

The appropriate number of packed cartons required for each in-use study (17 Day, 34 Day, 200 Day & 24 Hour Temperature Excursion) were delivered to the laboratory at “Time 0 days” and were set down at 5° C. #3° C. in the appropriate orientation as indicated on the label; (U)=Upright, (I)=Inverted.

### 6. Conclusion

The data presented above support the registered shelf life of 24 months for Phenelzine film coated tablets when stored between 2° C. to 8° C. in a refrigerator, in its original packaging. In-use stability studies were performed at 17 days, 34 days and 200 days on samples stored (upright & inverted) at 5° C. #3° C. A 24 Hour Temperature Excursion Study was also performed on samples stored (upright & inverted) at 5° C.±3° C.

All studies simulated the use of a multi-dose container by the patient. Product quality was assessed by performing analytical testing on the samples before and after storage of the container incorporating regular removal of tablets and trending of the results to ensure they meet specification over the course of each in-use study.

All test results have passed specification at the initial and end date for the 17 Day Study, 34 Day Study, 200 Day Study and 24 Hour Temperature Excursion Study. As such, these studies have been completed successfully and support the in-use stability of Phenelzine 15 mg tablets to support the overall stability study of Phenelzine.

1. An oral dosage form comprising:
(a) a pharmaceutical composition comprising phenelzine or a pharmaceutically acceptable salt thereof, in combination with one or more pharmaceutically acceptable excipients; and
(b) a film coating surrounding said pharmaceutical composition;
said wherein the film coating is an aqueous based film coating.

2-26. (canceled)

27. The oral dosage form according to claim 1, wherein said oral dosage form is a tablet or a capsule.

28. The oral dosage form according to claim 1, wherein the phenelzine is phenelzine sulfate.

29. The oral dosage form according to claim 28, wherein the phenelzine sulfate is present within the oral dosage form in a range of between 10 mg and 90 mg per oral dosage form.

30. The oral dosage form according to claim 1, wherein the pharmaceutical composition comprises a diluent.

31. The oral dosage form according to claim 30, wherein the diluent is mannitol, which is present within the oral dosage form in a range of between 150 mg and 250 mg per oral dosage form.

32. The oral dosage form according to claim 1, wherein the pharmaceutical composition comprises a disintegrant.

33. The oral dosage form according to claim 32, wherein the disintegrant is povidone which is present within the oral dosage form in a range of between 1 mg and 10 mg per oral dosage form.

34. The oral dosage form according to claim 1, wherein the pharmaceutical composition comprises a lubricant.

35. The oral dosage form according to claim 34, wherein the lubricant is magnesium stearate, which is is present within the oral dosage form in a range of between 1 mg and 10 mg per oral dosage form.

36. The oral dosage form according to claim 1, wherein the pharmaceutical composition comprises a binder.

37. The oral dosage form according to claim 36, wherein the binder is maize starch, which is present within the oral dosage form in a range of between 1 mg and 10 mg per oral dosage form.

38. The oral dosage form according to claim 1, wherein the film coating comprises: polyvinyl alcohol, talc, glyceryl dicaprylocaprate, glyceryl monocaprylocaprate, sodium lauryl sulfate, and titanium dioxide.

39. The oral dosage form according to claim 38, wherein the polyvinyl alcohol is present within the film coating in an amount of between 1 mg and 10 mg per oral dosage form.

40. The oral dosage form according to claim 38, wherein the talc is present within the film coating in an amount of between 1 mg and 10 mg per oral dosage form.

41. The oral dosage form according to claim 38, wherein the combination of glyceryl dicaprylocaprate and glyceryl monocaprylocaprate is present within the film coating in an amount of between 0.5 mg and 5 mg per oral dosage form.

42. The oral dosage form according to claim 38, wherein the sodium lauryl sulfate is present within the film coating in an amount of between 0.01 mg and 2 mg per oral dosage form.

43. The oral dosage form according to claim 38, wherein the titanium dioxide is present within the film coating in an amount of between 0.1 mg and 1 mg per oral dosage form.

44. The oral dosage form according to claim 1, wherein the film coating is a coloured coating.

45. The oral dosage form according to claim 44, wherein the coloured coating comprises sunset yellow and carmine.

46. The oral dosage form according to claim 45, wherein the sunset yellow is present within the film coating in an amount of between 1 mg and 5 mg per oral dosage form and the carmine is present within the film coating in an amount of between 0.01 mg and 2 mg per oral dosage form.

47. The oral dosage form according to claim 1, wherein the film coating is provided within the oral dosage form in an amount of between 5 mg and 25 mg per oral dosage form.

48. The oral dosage form as described in Tables 1 and 2:
TABLE 1
Composition of Drug Product

| | Quantity | | Reference |
|---|---|---|---|
| Ingredient | (mg/tab) | Function | Standard |
| Active Substance | | | |
| Phenelzine Sulfate | 25.83 | Active | BP |
| (Equivalent to | | | |
| Phenelzine base 15 mg) | | | |
| Excipients | | | |
| Mannitol | 185.96 | Diluent | Ph. Eur. |
| Povidone | 6.83 | Disintegrant | Ph. Eur. |
| Magnesium Stearate | 6.81 | Lubricant | Ph. Eur. |
| Maize Starch | 4.57 | Binder | Ph. Eur. |
| Isopropyl alcohol | ND | Solvent | Ph. Eur. |
| Film Coating | | | |
| Opadry AMB II Orange | 13.80 | Coating | See Table 2 |
| Water Purified | ND | Solvent | Ph. Eur. |

TABLE 2
Composition of Opadry AMB II Orange

| | Quantity | % | | |
|---|---|---|---|---|
| Ingredient | (mg/tab) | w/w | Function | Reference Standard |
| Polyvinyl | 5.45 | 39.5 | Coating | USP, FCC, Ph. Eur, |
| Alcohol | | | | JPE, ChP, GB |
| Talc | 5.24 | 38.0 | Coating | USP, FCC, Ph. Eur, |
| | | | | JP, JECFA |
| Sunset Yellow | 1.52 | 11.0 | Coating | JECFA, JSFA, JP |
| FCF | | | | |
| Aluminum Lake | | | | MO, GB |
| Glyceryl | 0.62 | 4.5 | Coating | NF, Glycerol of FA |
| Dicaprylocaprate | | | | JSFA |
| Glyceryl Mono- | | | | Type 1 Ph. Eur, |
| caprylocaprate | | | | Glycerol of FA |
| | | | | JSFA |
| Sodium Lauryl | 0.41 | 3.0 | Coating | NF, Ph. Eur, JP, |
| Sulfate | | | | ChP 2015 |
| Titanium | 0.41 | 3.0 | Coating | USP, FCC, Ph. Eur, |
| Dioxide | | | | JP, JSFA, ChP, GB |
| Carmine | 0.14 | 1.0 | Coating | FCC, JPE, JECFA |

49. A method of treating depression, anxiety, or a disorder related to depression or anxiety in a subject comprising administering the oral dosage form according to claim 1 to a subject in need thereof.

50. The method of claim 49, wherein the depression is major depressive disorder (MDD).

51. A method of treating dysthymia, bipolar depression (BD), panic disorder (PD), social anxiety disorder, bulimia, post-traumatic stress disorder (PTSD), and obsessive-compulsive disorder (OCD) in a subject comprising administering the oral dosage form according to claim 1 to a subject in need thereof.
